(19) United States
(12) Patent Application Publication
(10) Pub. No.: US 2021/0106518 A1
(43) Pub. Date: Apr. 15, 2021

(54) U-SHAPED ORAL THIN FILM

(71) Applicant: LTS LOHMANN THERAPIE-SYSTEME AG, Andernach (DE)

(72) Inventors: Christoph SCHMITZ, Rheinbrohl (DE); Marius BAUER, Andernach (DE); Michael LINN, Waldböckelheim (DE)

(21) Appl. No.: 17/050,183

(22) PCT Filed: Apr. 25, 2019
(86) PCT No.: PCT/EP2019/060665

§ 371 Date: Oct. 23, 2020

(30) Foreign Application Priority Data
Apr. 25, 2018 (DE) 10 2018 109 981.9

(51) Int. Cl. A61K 9/00; A61K 47/38; A61K 47/32; A61K 9/70

(52) U.S. Cl. CPC A61K 9/006; A61K 47/38; A61K 31/135; A61K 9/7015; A61K 47/32

(57) ABSTRACT

The present invention relates to an oral thin film comprising at least one polymer and at least one pharmaceutically active ingredient, the oral thin film being substantially U-shaped, and to the use of said oral thin film as a medicament, especially for sub-lingual administration.

The present invention relates to a U-shaped oral thin film and to its use as a medicament, especially for sublingual administration.

Oral thin films are thin films containing at least one pharmaceutically active ingredient which are placed directly in the oral cavity or are placed against the oral mucosa and dissolve there. Especially, they are thin active-ingredient-containing, polymer-based films, which, when applied to a mucosa, especially the oral mucosa, deliver the active ingredient directly into the mucosa. Especially, the oral thin film can be administered in the space or on the mucosa beneath the tongue (sublingual administration). The good vascular supply and very good blood supply to the oral mucosa in this area ensures a rapid absorption of the active ingredient.

This dosage form has the advantage that the active ingredient is absorbed for the most part by the mucosa, and therefore the first-pass metabolism, which occurs with active ingredients provided in the conventional tablet dosage form, is avoided. The active ingredient may be dissolved, emulsified or dispersed in the film.

The oral thin films known from the prior art, including those intended for sublingual administration, are almost exclusively provided in, rectangular form, since this shape is the simplest and most economical to produce. Oral thin films for sublingual administration which are substantially rectangular, but which have a small recess to accommodate the “frenulum linguae” (lingual frenulum) in order to increase the level of comfort when the film is placed under the tongue are also known.

U.S. Pat. No. 8,475,832 A1, for example, discloses a rectangular oral thin film comprising buprenorphine as the active ingredient, amongst other things for sublingual administration.

WO 2013173697 A1 discloses oral thin films in strip form for use in the treatment of allergies.

These oral thin films known from the prior art have the disadvantage that the available space under the tongue is not optimally utilised. This is especially disadvantageous when administering active ingredients that are to be administered sublingually in a relatively high dose. Since the weight per unit area and thus the quantity of active ingredient per unit area in an oral thin film is limited by many factors (such as drying time, dissolution time of the oral thin film, etc.) it is therefore necessary to make the best possible use of the available space. This is not the case with substantially rectangular oral thin films for sublingual administration.

The aim of the present invention is to remedy the aforementioned disadvantages of the prior art. Especially, the aim of the present invention is to provide an oral thin film which makes optimum use of the space under the tongue, especially in order to administer also large quantities of active ingredients. In addition, the oral thin film should also be economical, i.e. it should be possible to produce it without much material loss.

The above aim is addressed by an oral thin film according to claim 1, i.e. by an oral thin film comprising at least one polymer and at least one pharmaceutically active ingredient, characterised in that the oral thin film is substantially U-shaped.

Such a U-shaped oral thin film is characterised in that it makes optimum use of the available space under the tongue. Especially, by means of the legs of the U-shaped oral thin film, optimum use is made of the space under the tongue from the region of the front incisors to the back in the region of the tongue root. In addition, such a U-shaped oral thin film can be administered very comfortably, since on the one hand it is recessed to accommodate the frenulum linguae and on the other hand there are no sharp corners or points that could negatively influence comfort once the film has been placed under the tongue. In addition, such a U-shaped oral thin film has the advantage that, if arranged appropriately, it can be punched out or cut out of a large-area film without much loss.

### DESCRIPTION OF THE DRAWINGS

FIG. 1 shows a schematic view of forms that are subsumed under the term “U-shaped” used in accordance with the invention. These are merely limiting forms, and therefore all forms in between also fall under the term “U-shaped”.

FIG. 2 shows a schematic view of a U-shaped oral thin film according to the invention.
(1) total length of oral thin film
(2) total width of the oral thin film
(3) length of the substantially parallel ends
(4) width of the oral thin film in the region of the substantially parallel ends
(4a) width of the oral thin film in the region of the join
(5) length of the oral thin film in the region of the join

FIG. 3 shows one way in which U-shaped oral thin films according to the invention can be cut out or punched out from a template without much material loss, despite their relatively complex shape.

The oral thin film according to the invention comprises at least one polymer. Suitable polymers are substantially, without restriction, all polymers known to a person skilled in the art for producing oral thin films.

The oral thin film according to the invention additionally comprises at least one pharmaceutically active ingredient. The active ingredient can be selected, without restriction, from all pharmaceutically active ingredients suitable for oral and/or transmucosal administration.

The oral thin film according to the invention is characterised especially in that it is substantially U-shaped. The term “U-shaped” means that the oral thin film is substantially similar to the letter “U” of the Latin alphabet.

The term “U-shaped” is therefore understood to mean a shape in which two substantially parallel ends are joined together by a join at one end on the same side. This join can be made possible by a curved join, so that a “round” U is formed. However, it can also be made by a straight joint to form an angular “U”.

Furthermore, the term “U-shaped” shall also include embodiments of the oral thin film according to the invention in which the join of the two substantially parallel ends on the same side is formed by two legs which meet at an apex, i.e. a kind of triangle without a base. The result is a pointed, angular “V”.

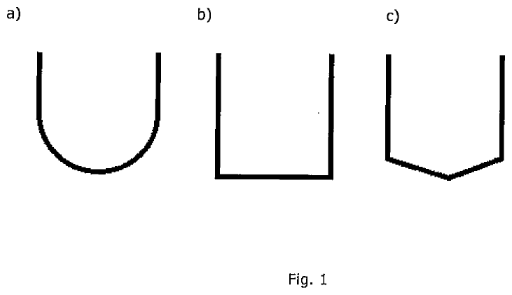

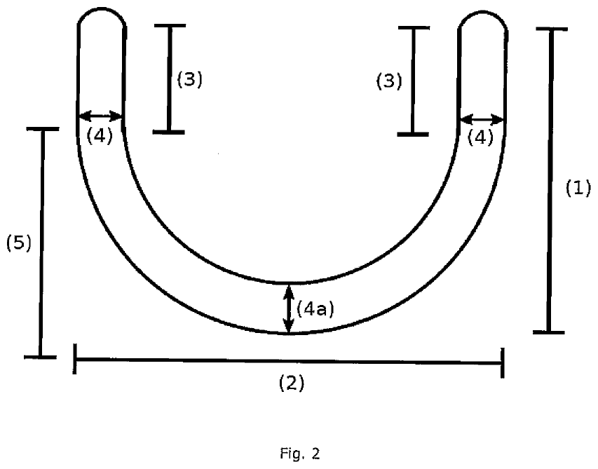

Examples of forms as described above and which fall under the term “U-shaped” are shown in FIG. 1.

Specifically, the “round” U is shown in FIG. 1a, the “angular” U is shown in FIG. 1b, and the “pointed angular” U is shown in FIG. 1c.

However, these specifically described forms are merely limiting forms, and therefore forms in between also fall under the term “U-shaped”.

In the following, preferred embodiments of the “U-shaped” oral thin film according to the invention will be described.

The dimensions shown below with the corresponding reference signs refer to the “round U” embodiment. However, these are also to be applied analogously to the other defined forms, such as the “angular U” and the “pointed angular U”.

The U-shaped oral thin film has a curved region over the entire width (2) of the oral thin film, which curved region also extends over a part (5) of the total length (1) of the oral thin film, and two ends, which each follow on from a join. The joins (2), (5) of the oral thin film are preferably designed in such a way that outgoing ends (3) are substantially parallel.

The oral thin film according to the invention is preferably characterised in that the two ends (3) run substantially parallel to each other. Preferably, all four edges, i.e. the two inner edges as well as the two outer edges of the oral thin film, run substantially parallel to each other.

The expression “substantially parallel” is understood to mean that the edges may deviate from an imaginary exactly parallel arrangement in both directions in the plane of the surface from 0° to approximately 15°.

The substantially parallel arrangement of the ends (3) has the advantage that the spaces in the rear region under the tongue, i.e. towards the root of the tongue, can also be optimally utilised.

Preferably, the corners of the ends (3) are slightly rounded, thus increasing comfort when the film is placed under the tongue.

The oral thin film according to the invention is also preferably characterised in that it has a substantially constant width (4), (4a) in the region of the substantially parallel ends and in the region of the join.

The width (4) is understood to mean the width of the actual surface of the oral thin film in the region of the parallel ends. The width (4a) is understood to mean the width of the actual surface of the oral thin film in the region of the join (2), (5), which is defined by the total width of the oral thin film (2) and the length of the join (5).

The substantially constant width (4), (4a) in the region of the substantially parallel ends and in the region of the join is preferably approximately 0.6 to 1.4 cm.

A substantially constant width (4), (4a) has the advantage that the oral thin film dissolves evenly and thus releases the active ingredient constantly.

The oral thin film according to the invention is furthermore preferably characterised by the fact that it has a total length (1) of approximately 2.0 to 4.0 cm.

The oral thin film is also preferably characterised in that it has a total width (2) of approximately 3.0 to 4.5 cm.

In some embodiments it is preferred that the width (4a) in the region of the join (2), (5) of the oral thin film according to the invention is slightly larger than the width (4) in the region of the substantially parallel ends (3). It is preferred that the oral thin film, in the region of the join (2), (5), has a width (4a) which is 0.01 to 20% greater than the width (4) in the region of the substantially parallel ends (3).

This has the advantage that the space under the tongue can be even better utilised, which is especially advantageous when administering relatively large quantities of active ingredients.

The total length (1) is the sum of the length of the join (5) and the length of the substantially parallel ends (3). The length of the join (5) and the length of the substantially parallel ends (3) can be adjusted independently of one another to provide oral thin films that accommodate different jaw sizes and jaw shapes of patients.

The oral thin film according to the invention is preferably characterised in that the join (5) has a total length of approximately 1.5 to 2.4 cm (?).

The oral thin film according to the invention is furthermore preferably characterised in that the substantially parallel ends (3) each have a total length of approximately 0.1 to 1.5 cm.

The oral thin film according to the invention is further preferably characterised in that the total area of a surface of the oral thin film according to the invention is 1 to 10 cm².

Preferably, the oral thin film according to the invention has a thickness of up to 1 mm. However, embodiments of the oral thin film according to the invention in which the oral thin film according to the invention has a thickness of up to 3 mm are also possible.

The oral thin film according to the invention is preferably designed for administration to adult patients. If such an oral thin film were to be provided for administration to babies or children, the size of the oral thin film, i.e. the total length (1), the total width (2), the length of the join (5) and the length of the substantially parallel ends (3), as well as the width (4) and (4a), must be adapted to the jaw sizes of the babies or children, which is easily possible for a person skilled in the art.

It is preferred that the edges and corners of a U-shaped oral thin film according to the invention, especially in the case of the “angular” and the “angular pointed” U, are each rounded.

The oral thin film in the form of the “round U” is especially preferred since it has the fewest sharp edges andor corners.

The oral thin film according to the invention comprises a polymer, the polymer preferably being a water-soluble andor water-swellable polymer.

Water-soluble andor water-swellable polymers comprise chemically very different, natural or synthetic polymers of which the common feature is their solubility or swellability in water or aqueous media.

Preferably, the oral thin film according to the invention is characterised in that the polymer is selected from the group consisting of starch and starch derivatives, dextrans; cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose; polyacrylic acids, polyacrylates, polyvinylpyrrolidones, polyvinyl alcohols, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan and natural gums, this group not being exhaustive.

The weight per unit area of the oral thin film is preferably approximately 20 to 300 g/m², especially preferably approximately 100 to 250 g/m².

The at least one pharmaceutically active ingredient contained in the oral thin film according to the invention is, in principle, not subject to any restriction, but is preferably selected from all pharmaceutically active ingredients suitable for oral andor transmucosal application.

Preference is given to active ingredients selected from the group consisting of the active ingredient classes of analgesics, hormones, hypnotics, sedatives, antiepileptics, wake-up amines, psychoneurotropic agents, neuro-muscle blockers, antispasmodics, antihistamines, antiallergics, cardiotonics, antiarrhythmics, diuretics, hypotensives, vasopressors, antidepressants, antitussives, expectorants, thyroid hormones, sex hormones, antidiabetics, anti-tumour active ingredients, antibiotics, chemotherapeutic agents and narcotics, this group not being exhaustive.

In an especially known embodiment, ketamine is used as the active ingredient, preferably in a quantity of approximately 20 to 40% by weight. In this embodiment, hydroxypropyl methylcellulose or polyvinyl alcohol is especially preferably used as polymer.

The quantity of active ingredient in the oral thin film according to the invention is dependent on the type of active ingredient and is usually approximately 0.01 and 70% by weight.

Preferably, the oral thin film according to the invention also comprises at least one auxiliary selected from the group comprising dyes, flavourings, sweeteners, taste-masking agents, surfactants, enhancers, pH regulators, preservatives, antioxidants andor plasticisers.

These auxiliaries are preferably each contained in the oral thin film according to the invention in a quantity of from 0.001 to 20% by weight.

The oral thin film according to the invention is preferably a single-layer U-shaped oral thin film. However, embodiments are also possible in which the oral thin film according to the invention is a multi-layer U-shaped oral thin film. The individual layers can be joined together by means of coating, laminating, sealing or adhesives. In such multi-layer oral thin film, each individual layer may contain the same or different pharmaceutically active ingredients.

The present invention also relates to the use of the oral thin film according to the invention as a medicinal product, especially for sublingual application.

The oral thin film according to the invention can be produced according to the usual methods for the production of oral thin films. For example, such a method includes the steps:
a) preparing a suspension or solution comprising the at least one polymer and the at least one pharmaceutically active ingredient;
b) spreading and drying the suspension or solution obtained in step a) so that a thin film, preferably with a weight per unit area of from 20 to 350 g/m² is obtained; and
c) cutting out or punching out the U-shaped oral thin film from the thin film obtained in step b).

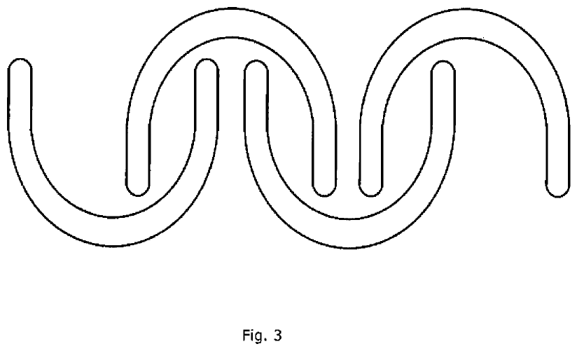

The U-shape of the oral thin film according to the invention has the advantage that, despite the relatively complex shape, it can be cut out or punched out of the thin film without much loss in step c) of the method described above. This is shown schematically for four U-shaped oral thin films in the form of the “round” U in FIG. 3.

1. Oral thin film comprising at least one polymer and at least one pharmaceutically active ingredient, characterised in that the oral thin film is substantially U-shaped.

2. Oral thin film according to claim 1, characterised in that the oral thin film is constructed in such a way that two ends are substantially parallel,

3. Oral thin film according to claim 2, characterised in that the oral thin film has a substantially constant width in a region of the substantially parallel ends and, in a region of a join has a constant width of from 0.6 to 1.4 cm.

4. Oral thin film according to claim 1, characterised in that the oral thin film has a total length from 2.0 to 4.0 cm.

5. Oral thin film according to claim 1, characterised in that the oral thin film has a total width from 3.0 to 4.5 cm.

6. Oral thin film according to claim 2, characterised in that the oral thin film has a width in a region of a join which is 0.01-20% greater than a width in the region of the substantially parallel ends.

7. Oral thin film according to claim 1, characterised in that a join has a total length of from 1.5 to 2.4 cm.

8. Oral thin film according to claim 2, characterised in that the substantially parallel ends each have a total length of from 0.1 to 1.5 cm.

9. Oral thin film according to claim 1, characterised ire that a total area of a sutlace of the oral thin film is 1 to 10 cm².

10. Oral thin film according to claim 1, characterised in that, the polymer is a water-soluble andor water-swellable polymer.

11. Oral thin film according to claim 1, characterised in that the polymer is selected from the group consisting of starch and starch derivatives, dextrans, cellulose derivatives such as carboxymethyl cellulose, hydroxypropyl cellulose, hydroxyethyl cellulose, hydroxypropylmethyl cellulose, hydroxypropyl ethyl cellulose, sodium carboxymethyl cellulose, ethyl or propyl cellulose, polyacrylic acids, polyacrylates, polyvinylpyrrolidones, polyvinyl alcohols, polyethylene oxide polymers, polyacrylamides, polyethylene glycols, gelatine, collagen, alginates, pectin, pullulan, tragacanth, chitosan, alginic acid, arabinogalactari, galactomartnan, agar, agarose, carrageenan and natural gums.

12. Oral thin film according to claim 1, characterised in that the oral thin film has a weight per unit area of from 20 to 350 g/m².

13. Oral thin film according to claim 1, characterised in that the pharmaceutically active ingredient is selected from the group consisting of analgesics, hormones, hypnotics, sedatives, antiepileptics, wake-up amines, psychoneurotropic agents, neuro-muscle blockers, antispasmodics, antihistamines, antiallergios, cardiotonics, antiarrhythmics, diuretics, hypotensives, vasopressors, antidepressants, antitussives, expectorants, thyroid hormones, sex hormones, antidiabetics, anti-tumour active ingredients, antibiotics, chemotherapeutic agents and narcotics.

14. Oral thin film according to claim 1, characterised in that the oral thin film contains at least one auxiliary selected from the group comprising dyes, flavourings, sweeteners, taste-masking agents, surfactants, enhancers, pH regulators, preservatives, antioxidants andor plasticisers.

15. A method of supplying a medicament to a patient comprising administering the oral thin film according to claim 1.

16. The method of claim 15, wherein the oral thin film is administered sublingually.
